(19) United States
(12) Patent Application Publication
(10) Pub. No.: US 2019/0321487 A1
(43) Pub. Date: Oct. 24, 2019

(54) CYCLODEXTRIN CONJUGATES

(71) Applicant: UNIVERSITY COLLEGE CORK - NATIONAL UNIVERSITY OF IRELAND, CORK, Cork (IE)

(72) Inventors: Raphael DARCY, Dublin (IE); Caitriona O'DRISCOLL, Cork (IE); Meenakshi MALHOTRA, Palo Alto, CA (US)

(73) Assignee: UNIVERSITY COLLEGE CORK - NATIONAL UNIVERSITY OF IRELAND, CORK, Cork (IE)

(21) Appl. No.: 16/469,248

(22) PCT Filed: Dec. 14, 2017
(86) PCT No.: PCT/EP2017/082941

§ 371 Date: Jun. 13, 2019

(30) Foreign Application Priority Data
Dec. 14, 2016 (EP) 16204184.2

(51) Int. Cl. A61K 47/69; A61K 47/62; A61K 47/61; A61K 31/7105

(52) U.S. Cl. CPC A61K 47/6951; A61K 47/62; A61K 47/6929; A61K 31/7105; A61K 47/61

(57) ABSTRACT

A cyclodextrin-RNA conjugate is described in which the cyclodextrin molecule is conjugated at its glucosyl 6-, 2- or 3-positions, optionally via a linker, to at least one RNA molecule at the RNA 3′ terminal base. The at least one RNA molecule may be an siRNA molecule.

### TECHNICAL FIELD

This invention provides cyclodextrin conjugates, and the use of cyclodextrin conjugates to deliver nucleic acid to a cell in-vivo, in-vitro, or ex-vivo. The invention also relates to nanoparticulate molecular complexes formed with the cyclodextrin conjugates, and the use of such complexes for therapeutic purposes, alone or formulated with pharmaceutical ingredients.

### BACKGROUND TO THE INVENTION

Oligonucleotides have great potential as a new generation of therapeutics. The advent of RNA interference, along with that of antisense and catalytic RNA, has led to the prospect of controlling gene expression as a remedy for hereditary diseases, cancer, and infectious diseases. mRNA has potential in the form of genetic vaccines, through both in vivo administration and the treatment of dendritic cells for cellular generation of vaccines.

Yet the application of RNA in therapies is greatly hindered by problems with pharmaceutical delivery. RNAs are polyanionic macromolecules, and to reach their site of action they must penetrate the negatively charged outer cell plasma membrane and the lipophilic barrier of the cell membrane. Delivery of RNA into the cytoplasm is necessary to enable the endogenous biology to effect therapy.

Cationic lipids and polymers provide the most common methods of overcoming electrostatic repulsion, by complexing with the RNA molecules and neutralising the negative charges. Conjugation of siRNAs to active ligands has also shown promise in facilitating delivery. This may involve conjugating siRNAs to small peptides that can penetrate cellular membranes, or to lipophiles such as cholesterol.

Solution properties of the conjugated moiety must still override those of the relatively large oligonucleotide, and this is not routinely possible. This requirement has led to the development of nanoparticulate complexes in which the combined properties of the constituent molecules override those of the RNA. Nanoparticulate inclusion of the RNA also protects it from degradation en route to its target. Here there remains however the problem of obtaining spontaneous assembly of the constituent molecules, having modified their structures with ligands which may, like the oligonucleotide, be relatively large and polar.

These demands have led to the selection of cyclodextrins as core molecules for the synthesis of mesomolecular (medium-sized) delivery agents for RNA. They are monodisperse oligosaccharides capable of accommodating design variations involving multiple charges and polar or lipidic groups without disruption of their inherent assembly properties. In previous patents we have described such nanoparticles which protect the RNA from degradation, are amenable to coating with ligands for targeting to specific cell types or organs, and deliver the RNA cargo into cells to give knockdown of the appropriate messenger RNA (EP 1287039, U.S. Pat. No. 7,786,095; EP 2303929, US 2011/0124103). The modified cyclodextrins which are the subject of these patents form molecular complexes which adopt the nanoparticulate structure that encapsulates the RNA. These modified cyclodextrins are capable themselves of forming bilayers or vesicles in aqueous solutions. The modifications which are possible while maintaining these useful liquid crystalline properties are many, such as: incorporation of cationic groups to neutralise the anionic charges of RNA and promote complexation; the chemical linkage of various ligands for targeting the nanoparticles to particular cell types or molecules; coating of the nanoparticles with ligands by molecular inclusion; linkage of polyethylene glycol (PEG) groups for masking of electrostatic surface charge and as linkers for various ligands.

It is well known that cyclodextrins can be modified with hydrophilic groups and lipophilic groups at certain positions to make them amphiphilic. The resultant derivatives are generally capable of forming types of assemblies expected of amphiphiles. The successful applications of these amphiphilic cyclodextrins to delivery of siRNA are described in numerous scientific papers: Fitzgerald K A., Malhotra M., Gooding M., Sallas F., Evans J C., Darcy R., O'Driscoll C M. (2016) A novel, anisamide-targeted cyclodextrin nano formulation for siRNA delivery to prostate cancer cells expressing the sigma-1 receptor. Int. J., Pharm., 499:131-145; Fitzgerald K A., Guo G., Tierney E G., Curtin C M., Malhotra M., Darcy R., O'Brien F J., O'Driscoll C M. (2015) The use of collagen-based scaffolds to simulate prostate cancer bone metastases with potential for evaluating delivery of nanoparticulate gene therapeutics. Biomaterials, 66:53-66; Evans J C., McCarthy J., Torres-Fuentes C., Cryan J F., Ogier J., Darcy R., Watson R W., O'Driscoll C M. (2015) Cyclodextrin mediated delivery of NF-κB and SRF siRNA reduces the invasion potential of prostate cancer cells in vitro. Gene Therapy, 22 (10):802-810; Gooding M., Malhotra M., McCarthy D J., Godinho BMDC., Cryan J F., Darcy R., O'Driscoll C M. (2015) Synthesis and characterization of rabies virus glycoprotein-tagged amphiphilic cyclodextrins for siRNA delivery in human glioblastoma cells: in-vitro analysis. Eur J Pharm., Sci., 71:80-92; Godinho BMDC, Ogier J R, Quinlan A, Darcy R, Griffin B T, Cryan J F, O'Driscoll C M. (2014) PEGylated cyclodextrins as Novel siRNA nanosystems: correlations between polyethylene glycol length and nanoparticle stability. Int. J. Pharm., 473:105-112; O'Mahony A M., Cronin M F., McMahon A., Evans J C., Daly K., Darcy R., O'Driscoll C M. (2014) Biophysical and Structural Characterisation of Nucleic Acid Complexes with Modified Cyclodextrins Using Circular Dichroism. J. Pharm., Sci., 103:1346-1355; Hibbitts A, O'Mahony A M, Forde E, Nolan L, Ogier J, Desgranges S, Darcy R MacLoughlin R, O'Driscoll C M, Cryan S A. (2014) Early-Stage Development of Novel Cyclodextrin-siRNA Nanocomplexes Allows for Successful Postnebulization Transfection of Bronchial Epithelial Cells. Journal of Aerosol Medicine & Pulmonary Drug Delivery, 27(6): 466-477; Godinho BMDC., McCarthy D J., Torres-Fuentes C., Beltran C J., McCarthy J., Quinlan A., Ogier J R., Darcy R., O'Driscoll C M., Cryan J F. (2014) Differential nanotoxicological and neuroinflammatory liabilities of non-viral vectors for RNA interference in the central nervous system. Biomaterials, 35:489-499; O'Neill M., O'Mahony A., Byrne C., Darcy R., O'Driscoll C M. (2013) Gastrointestinal gene delivery by Modified Cyclodextrins—in vitro identification and quantification of extracellular barriers. Int J Pharmaceutics, 456:390-399; O'Mahony A M, Ogier J, Darcy R, Cryan J F, O'Driscoll C M. (2013) Cationic and PEGylated amphiphilic Cyclodextrins: Co-formulation opportunities for neuronal siRNA delivery. PLoS ONE 8(6): e66413; McCarthy J., O'Neill M J., Bouree L., Walsh D P., Quinlan A., Hurley G., Ogier J., Shanahan F., Melgar S., Darcy R and O'Driscoll C M. (2013) Gene silencing of TNF-alpha in a murine model of acute colitis using a modified cyclodextrin delivery system. J. Cont., Rel., 168:28-34; Villari, V., Mazzaglia, A., Darcy, R., O'Driscoll, C M and Micali, N. (2013) Nanostructures of cationic amphiphilic cyclodextrin complexes with DNA. Biomacromolecules, 14:811-817; Godinho BMDC, Ogier J R, Darcy R, O'Driscoll C M, Cryan J F. (2013) Self-assembling Modified Beta-Cyclodextrin Nanoparticles as Neuronal siRNA Delivery Vectors: Focus on Huntington's Disease. Molecular Pharmaceutics 10:640-649; O'Mahony, A M., Desgranges S., Ogier J R., Quinlan A., Devocelle M., Darcy, R., Cryan, J F and O'Driscoll, C M. (2013) In vitro investigations of the efficacy of Cyclodextrin-siRNA complexes modified with Lipid-PEG-Octaarginine: Towards A Formulation Strategy for Effective Neuronal siRNA Delivery. Pharm. Res., 30, 4:1086-1098; O'Mahony, A M, Doyle, D, Darcy, R, Cryan, J F, O'Driscoll, C M. (2012) Characterisation of cationic amphiphilic cyclodextrins for neuronal delivery of siRNA: effect of reversing primary and secondary face modifications. Eur. J. Pharm., Sci., 47:896-903; O'Mahony A M, Godinho BMDC, Ogier J, Devocelle M, Darcy R, Cryan J F, O'Driscoll C M. (2012) Click-modified cyclodextrins as non-viral vectors for neuronal siRNA delivery. ACS Chemical Neuroscience, 10(3):744-52; Guo J, Ogier J, Desgranges S, Darcy R, O'Driscoll C M. (2012) Anisamide-targeted cyclodextrin nanoparticles for siRNA delivery to prostate tumours in mice. Biomaterials, 33: 7775-7784; O'Mahony A M, Ogier J, Desgranges S, Cryan J F, Darcy R, O'Driscoll C M. (2012) A click chemistry route to 2-functionalised PEGylated and cationic Beta-cyclodextrins: co-formulation opportunities for siRNA delivery. Organic & Biomolecular Chemistry 10:4954-496; McMahon A, O'Neill M J, Gomez E, Donohue R, Forde D, Darcy R, O'Driscoll C M. (2012) Targeted gene delivery to hepatocytes with galactosylated amphiphilic cyclodextrins, J. Pharmacy and Pharmacology, 54, 1063-1073; O'Neill M J, Guo J, Byrne C, Darcy R and O'Driscoll C M. (2011) Mechanistic studies on the uptake and intracellular trafficking of novel cyclodextrin transfection complexes by intestinal epithelial cells. Int. J. Pharmaceutics, 413:174-183; McMahon A, Gomez E, Donohue R, Forde D, Darcy R and O'Driscoll C M. (2008) Cyclodextrin gene vectors: cell trafficking and the influence of lipophilic chain length. J. Drug Del. Sci. Tech. 18 (5):303-307; Cryan S A, Holohan A, Donohue R, Darcy R, O'Driscoll C M. Cell transfection with polycationic cyclodextrin vectors. Eur. J. Pharm. Sci, 2004, 21:625-633; Cryan S A, Donohue R, Ravoo B J, Darcy R, O'Driscoll C M. Cationic cyclodextrin amphiphiles as gene delivery vectors. J. Drug Del. Sci. Tech. 2004, 14 (1): 57-62; O'Mahony A M, O'Neill M J, Godinho BMDC, Darcy, R, Cryan J F, O'Driscoll C M. (2013) Cyclodextrins for Non-Viral Gene and siRNA Delivery. Pharmaceutical Nanotechnology, 1:6-14; O'Driscoll C M and Darcy R. (2002) Cyclodextrin constructs for delivery of genotherapeutic agents. World Market Series, Business Briefing, PharmaTech, Ref Library: 1-5.

Of particular relevance to the present patent are the papers describing formation of nanoparticulate complexes where the amphiphilic cyclodextrin and RNA are co-complexed with at least one other modified cyclodextrin (O'Mahony A M, Ogier J, Darcy R, Cryan J F, O'Driscoll C M. (2013) Cationic and PEGylated amphiphilic Cyclodextrins: Co-formulation opportunities for neuronal siRNA delivery. PLoS ONE 8(6): e66413; O'Mahony A M, Ogier J, Desgranges S, Cryan J F, Darcy R, O'Driscoll C M. (2012) A click chemistry route to 2-functionalised PEGylated and cationic Beta-cyclodextrins: co-formulation opportunities for siRNA delivery. Organic & Biomolecular Chemistry 10:4954-496. Also relevant are nanoparticulate complexes comprising a cyclodextrin and RNA and an amphiphile which is not a cyclodextrin (O'Mahony, A M, Desgranges S, Ogier J R, Quinlan A, Devocelle M, Darcy R, Cryan J F, O'Driscoll C M. (2013) In vitro investigations of the efficacy of Cyclodextrin-siRNA complexes modified with Lipid-PEG-Octaarginine: Towards a Formulation Strategy for Effective Neuronal siRNA Delivery. Pharm. Res., 30, 4:1086-1098. Use of cyclodextrins as templates for the molecular constituents makes possible the incorporation of various chemical groups including ligands into the complex with RNA, since these comparatively smaller groups cannot disrupt self-assembly of the mesomolecular cyclodextrins. A cyclodextrin-RNA conjugate directly confers these self-assembly properties on the RNA as a means of enhancing its delivery.

### STATEMENTS OF INVENTION

The present invention derives from the discovery that a siRNA conjugated by means of a chemical linker to a cyclodextrin retains its ability to be effective in gene knockdown—the silencing of a gene. In order to study the co-complexation of variously modified cyclodextrins, a siRNA was conjugated to a cyclodextrin and the resulting CD-RNA conjugate incorporated into a nanoparticle with a cationic amphiphilic cyclodextrin as delivery vector. This nanoparticulate formulation delivered the CD-RNA conjugate to biological cells and resulted in ‘knockdown’ of the targeted gene. This result was unexpected due to the major modification made to the RNA, and it means that conjugation of an RNA to a cyclodextrin makes possible the formulation of RNA complexes in ways not previously designed and which are potentially more effective in therapeutic delivery.

In a first aspect, the invention provides a cyclodextrin-nucleic acid conjugate in which at least one nucleic acid is conjugated (typically via the 3′ terminal base) to the glucosyl units of the cyclodextrin at a glucosyl 6-, 2- or 3-position, optionally via a linker.

In one embodiment, the nucleic acid is selected from an RNA or DNA molecule. Examples of RNA molecules include mRNA (messenger RNA), siRNA (small interfering RNA), shRNA (small hairpin RNA), gRNA (guide RNA) or analogs thereof. Examples of DNA molecules include genes and transcriptional regulatory elements.

In one embodiment, the RNA is siRNA which is typically conjugated to the glucosyl unit via its 3′ terminal base.

In one embodiment, the cyclodextrin is amphiphilic (i.e. it is a modified cyclodextrin that is amphiphilic as a result of its modifications). Amphiphilic cyclodextrins, and conjugates formed therefrom, are capable of self-delivery into cells. Examples of amphiphilic cyclodextrins are described in the literature, for example US2006148756 and US2011124103. Other examples of amphiphilic cyclodextrins are described in Bilensoy et al (Expert Opin Drug Deliv 2009 November; 6(11)), Alejandro Daz-Moscoso, et al. (Polycationic Amphiphilic Cyclodextrins for Gene Delivery: Synthesis and Effect of Structural Modifications on Plasmid DNA Complex Stability, Cytotoxicity, and Gene ExpressionChem. Eur. J. 2009, 15, 12871-12888), and Pflueger et al. (Cyclodextrin-based facial amphiphiles: assessing the impact of the hydrophilic-lipophilic balance in the self-assembly, DNA complexation and gene delivery capabilities” Org Biol Chem 2016. DOI: 10.1039/c6ob01882c). Self-delivery may also be achieved by attaching a targeting ligand to the cyclodextrin.

In one embodiment, the amphiphilic cyclodextrin-nucleic acid conjugate of the invention has the following formula:

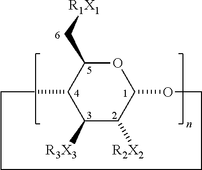

in which:
n equals 6 or 7 or 8, and indicates the number of modified or unmodified glucose units in the cyclodextrin macrocycle which are the same or different, depending on the X- and R-groups;
X₁, X₂, X₃ independently provide linkers and are a simple covalent bond or an atom or radical having a valency of at least two;
R₁, R₂ and R₃ independently are selected from the groups comprising (a) lipophilic groups, (b) polar groups and/or groups capable of hydrogen bonding, (c) targeting ligands, and (d) a nucleic acid molecule,
wherein at least one of R₁, R₂ and R₃ is a nucleic acid molecule.

In one embodiment, R₁ is a lipophilic group and R₂ and R₃ are independently selected from polar groups and/or groups capable of hydrogen bonding.

In one embodiment, the cyclodextrin has a primary side that is hydrophilic and comprises the R₁X₁ groups and a secondary side that is lipophilic and comprises the R₂X₂ and R₃X₃ groups, wherein R₁ and one of R₂ and R₃ are independently a polar group and/or group capable of hydrogen bonding, and in which the other of R₂ and R₃ is a lipophilic group.

In one embodiment, the groups that are polar and/or capable of hydrogen-bonding are selected from: H; OH; (CH₂)₂₋₄OH; CH₂CH(OH)CH₂OH; CH₂CH(OH)CH₂NH₂; an amine group; a cationic group; an anionic group; a polyamino acid; a peptide; an oligosaccharide; a polyethylene glycol (PEG) group; and a hydrophilic group.

In one embodiment, the targeting ligand is selected from a group comprising: a polyaminoacid, a peptide, an oligosaccharide, a steroid, —P(Y)(Z)O-nucleoside, —P(Y)(Z)O-oligonucleotide, —P(Y)(Z)O-Linker-OP(Y′)(Z)O-oligonucleotide, a nucleotide, an oligonucleotide, wherein Y, Z, Y′ and Z′ are independently O, or S.

In one embodiment, the lipophilic group is selected from an aliphatic chain, an alicyclic, aromatic or heterocyclic group, or combinations thereof.

In one embodiment, X₁, X₂, X₃ are independently selected from an ether, ester, methylene, methylenoxy, ethylene, ethylenoxy, carbonyl, thioether, thioester, thiocarbonyl, sulfanyl, disulfide, sulfonyl, sulfoxy, amido, amino, phosphate, thiophosphate, triazolyl.

In one embodiment, the amine group is selected from the group comprising NH₂, NHR where R is one or more of a methyl group, ethyl group, a branched or dendrimeric group comprising one to ten amine groups or a peptide containing basic amino acids, where one or more of the amino groups is optionally in its protonated form, preferably as a hydrohalide salt or trifluoroacetic acid salt.

In one embodiment, the polyethylene glycol (PEG) group is a straight-chain or branched group with one or more independently of a glycosyl, an oligosaccharide, a peptide, a glycopeptide, a protein, an antibody and/or a targeting group attached thereto.

In one embodiment, the nucleic acid is single-stranded. In one embodiment, the nucleic acid is double-stranded. In one embodiment, the RNA is single-stranded. In one embodiment, the RNA is double-stranded.

The invention also provides a nanoparticulate molecular complex comprising a cyclodextrin-RNA conjugate according to the invention. The complex may be formed from a self-assembling amphiphilic cyclodextrin-RNA conjugate according to the invention, or may require an additional complexing amphiphile (for example an amphiphilic cyclodextrin such as those described in US2006148756 and US2011124103), or a non-cyclodextrin amphiphile such as a PEG-lipid (polyethylene glycol-lipid conjugate, peptide-polyethylene glycol-lipid conjugate) or a cationic lipid such as N-[1-(2,3-dioleoyloxy)]-N,N,N-trimethylammonium propane methyl sulfate (DOTAP).

Thus, in one embodiment, the nanoparticulate molecular complex is self-assembling and is one in which the cyclodextrin-RNA conjugate is an amphiphilic cyclodextrin-RNA according to the invention.

In another embodiment, the nanoparticulate molecular complex comprises a complexing amphiphile.

In one embodiment, the complexing amphiphile is an amphiphilic cyclodextrin.

In one embodiment, the cyclodextrin-RNA conjugate is complexed with a polycation. In one embodiment, the polycation is selected from: a polycationic cyclodextrin, a polycationic lipid, a polycationic oligosaccharide, a polycationic polysaccharide, a polycationic peptide, a polycationic polymer.

In one embodiment, the nanoparticulate molecular complex according to the invention, incorporates additionally, by host-guest inclusion or by amphiphilic or electrostatic interaction, a lipophile such as a cholesterol or adamantane derivative and/or other lipid derivative.

In one embodiment, the nanoparticulate molecular complex according to the invention, is coated, by host-guest inclusion or by amphiphilic or electrostatic interaction, with a polyamine, peptide, protein, oligosaccharide, modified cyclodextrin, polysaccharide, antibody and/or antibody fragment.

The invention also relates to a method of delivering a functional RNA molecule to a cell, comprising a step of delivering to the cell a cyclodextrin-nucleic acid conjugate according to the invention or a nanoparticulate molecular complex according to the invention, wherein the cyclodextrin-nucleic conjugate comprises a functional RNA molecule.

The invention also relates to a method of modulating the expression (i.e. inhibiting the expression) of a target gene in a cell comprising a step of delivering to the cell a cyclodextrin-RNA conjugate of the invention or a nanoparticulate molecular complex of the invention, wherein the cyclodextrin-RNA conjugate comprises an RNA molecule that specifically targets a mRNA molecule corresponding to the target gene.

The invention also provides a pharmaceutical composition comprising the cyclodextrin-RNA conjugate of the invention, or a nanoparticulate molecular complex of the invention, alone or in combination with one or more pharmaceutically acceptable carriers or excipients.

For ease of exposition the complexes, formulations and methods are discussed here mainly with regard to conjugates formed between various cyclodextrins and unmodified siRNA agents. It should be understood however that these complexes, formulations and methods can also be practiced with modified siRNA agents, or with siRNA analogues, or with unmodified or modified RNA or agents analogous to an RNA which function in their biology differently from the above, such as mRNA agents, and such practices are within the scope of the invention.

The cyclodextrin-RNA conjugates of the present invention may be configured to self-assemble in aqueous solution either alone or when admixed with amphiphilic cyclodextrin derivatives or with lipid derivatives to form nanoparticulate complexes. These can enclose and shield the conjugates, and hence the RNA moiety of the conjugates, from undesired external interactions such as biological degradation and association with proteins, as well as providing the possibility of incorporating surface ligands for targeting to specific cell types or molecules. The complexes incorporating the CD-RNA conjugate may be used to deliver, in effect, more than one RNA: one or more of these being conjugated to a cyclodextrin (CD) and another or others being incorporated into the complex by interaction with another constituent such as a cationic cyclodextrin, amphiphilic cyclodextrin, cationic polymer, polyamine, or as a conjugate of a lipid which is incorporated by hydrophobic interaction.

In a preferred embodiment the cyclodextrin is chemically conjugated to the RNA by a linker moiety. Since the cyclodextrin molecules in natural form are cyclic oligosaccharides with no free 1-hydroxyl groups in their glucosyl units, attachment of the RNA generally takes place at a glucosyl 6-position where there is a free primary hydroxyl group, or at a 2- or 3-position where there are secondary hydroxyls, and not by utilising the reactivity of a 1-hydroxyl as in all prior art describing conjugation of carbohydrates, including oligosaccharides and polysaccharides, to RNA.

In another embodiment of the invention the cyclodextrin conjugated to the RNA is amphiphilic because it possesses lipid groups at the 6-positions, and polar groups at the 2-, 3-positions of the cyclodextrin's glucose units.

In another embodiment the cyclodextrin conjugated to the RNA is amphiphilic because of possessing lipidic groups at the 2-positions or 2-, 3-positions and polar groups at the 6-positions of the glucose units.

In particular embodiments of the invention, the lipidic groups occupying positions on the CD-RNA conjugate or on the amphiphilic cyclodextrins complexed with the CD-RNA conjugate comprise one or more hetero atoms selected from the group oxygen, nitrogen and sulfur. In further embodiments the lipophilic groups are selected from one or more of ethers, esters, amides, carbamates, ketones, thioethers, thioesters, thioketones, sulfanyl, disulfide, sulfonyl, sulfoxy, sulfones, and triazoles.

In another embodiment the cyclodextrin-RNA conjugate is complexed with a polycation such as a polycationic cyclodextrin or polycationic polymer which results in compaction of the RNA molecule and neutralisation of its polyanionic charge. As a result of this complexation with a polycation, entry into a biological cell is facilitated; also the polycation which is chosen can be an endosomolytic agent which promotes release of the CD-RNA or cleaved RNA from an endosome within the cell.

In another aspect of the invention the cyclodextrin moiety of the conjugate is chemically linked to multiple ligands, thus exploiting the ‘cluster effect’ (when the total affinity of multiple ligands for their receptors is greater than an attraction in direct proportion to their number).

In another embodiment the ‘cluster effect’ is exploited by having the CD-RNA conjugate complexed with a modified cyclodextrin which has multiple targeting ligands attached to it.

In another embodiment targeting ligands will be incorporated into the complex by attachment to lipohilic groups that are non-covalently included. This inclusion can either be into the cyclodextrin cavity of the CD-RNA conjugate or into bilayers formed by it, or into the cavities of, or bilayers formed by, cyclodextrins co-complexed with the CD-RNA conjugate.

According to the invention, there is also provided a surface-coated assembly formed from the self-assembly in aqueous solvent of the CD-RNA conjugate alone or with one or more amphiphilic cyclodextrins and/or other amphiphile, coated with a cholesterol, adamantyl and/or other lipid derivative included into the cyclodextrin cavity of the CD-RNA conjugate or into bilayers formed by it, or into the cavities of, or bilayers formed by, cyclodextrins co-complexed with the CD-RNA conjugate.

In another embodiment cholesterol, adamantyl or other lipophilic groups are attached to non-lipophilic groups such as PEG enabling attachment of these non-lipophilic groups to the assemblies. This complex provides two major advantages, namely a number of different molecules can be attached to the assembly surface in this manner, either directly or via PEG linkages, as targeting functions, an example being an antibody or a protein such as transferrin. Additionally, PEG or modified PEG attached in this manner will reduce toxicity and increase circulation time of the administered molecular assembly.

According to the invention, there is also provided a surface-coated assembly formed from the self-assembly in aqueous solvent of the CD-RNA conjugate alone or with one or more amphiphilic cyclodextrin and/or other amphiphile, coated with a polyamine, peptide, protein, oligosaccharide, polysaccharide, antibody and/or antibody fragment. Ideally, such a surface-coated assembly is formed by amphiphilic or electrostatic interaction.

The invention also provides a cyclodextrin-RNA conjugate of the invention or a nanoparticulate molecular complex of the invention, for use in a method of treating a disease in an individual by modulating the expression of a target gene in a cell involved in the pathogenesis of the disease, in which the cyclodextrin-RNA conjugate or nanoparticulate molecular complex is delivered to the cell, and comprises an RNA molecule that specifically targets the target gene to modulate expression of the gene (for example, increase expression, decrease expression, switch on gene expression, switch off gene expression). The disease may be any disease, including a proliferative disorder (i.e. a cancer), a brain disease, a neurological disease (i.e. a neurodegenerative disease), an immune disorder (for example an autoimmune disease), an inflammatory disorder (for example inflammatory bowel disease), a liver disease,

In one embodiment, the conjugate includes a targeting ligand configured to target the cell. In one embodiment, the disease is cancer and in which the cell is a cancer cell. In one embodiment, the targeting ligand is configured to target the cancer cell. In one embodiment, the disease is neurodegenerative disease and in which the cell is a cell of the peripheral or nervous system. In one embodiment, the targeting ligand is configured to target the cell of the peripheral or central nervous system. In one embodiment, the disease is a liver disease and in which the cell is a hepatic cell. In none embodiment, the targeting ligand is configured to target the hepatic cell. In one embodiment, the disease is an inflammatory disease of the gut and in which the cell is a gut epithelial cell. In one embodiment, the targeting ligand is configured to target the gut epithelial cell. 34. In one embodiment, the disease is an immune disease and in which the cell is a cell of the immune system, for example, lymphocyte, killer T cell, natural killer cell, helper T cell, gamma delta T cell, and B-lymphocyte. In one embodiment, the targeting ligand is configured to target a cell of the immune system.

The invention also provides a pharmaceutical comprising a cyclodextrin-RNA conjugate of the invention or a nanoparticulate molecular complex of the invention in combination with a suitable pharmaceutical excipient.

In particular embodiments, a pharmaceutical composition is prepared by at least one of the following methods: spray drying, lyophilization, vacuum drying, evaporation, fluid bed drying, or a combination of these techniques; or microfluidic techniques; or sonication with a lipid, freeze-drying, condensation and other conditions for molecular complex self-assembly.

### Definitions

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, poisoning or nutritional deficiencies.

As used herein, the term “treatment” or “treating” refers to an intervention (e.g. the administration of an agent to a subject) which cures, ameliorates or lessens the symptoms of a disease or removes (or lessens the impact of) its cause(s). In this case, the term is used synonymously with the term “therapy”.

Additionally, the terms “treatment” or “treating” refers to an intervention (e.g. the administration of an agent to a subject) which prevents or delays the onset or progression of a disease or reduces (or eradicates) its incidence within a treated population. In this case, the term treatment is used synonymously with the term “prophylaxis”.

As used herein, an “effective amount or a therapeutically effective amount of” an agent defines an amount that can be administered to a subject without excessive toxicity, irritation, allergic response, or other problem or complication, commensurate with a reasonable benefit/risk ratio, but one that is sufficient to provide the desired effect, e.g. the treatment or prophylaxis manifested by a permanent or temporary improvement in the subject's condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure.

As used herein, the term “cancer” should be taken to mean a cancer selected from the group consisting of: fibrosarcoma; myxosarcoma; liposarcoma; chondrosarcom; osteogenic sarcoma; chordoma; angiosarcoma; endotheliosarcoma; lymphangiosarcoma; lymphangioendotheliosarcoma; synovioma; mesothelioma; Ewing's tumor; leiomyosarcoma; rhabdomyosarcoma; colon carcinoma; pancreatic cancer; breast cancer; ovarian cancer; prostate cancer; squamous cell carcinoma; basal cell carcinoma; adenocarcinoma; sweat gland carcinoma; sebaceous gland carcinoma; papillary carcinoma; papillary adenocarcinomas; cystadenocarcinoma; medullary carcinoma; bronchogenic carcinoma; renal cell carcinoma; hepatoma; bile duct carcinoma; choriocarcinoma; seminoma; embryonal carcinoma; Wilms' tumor; cervical cancer; uterine cancer; testicular tumor; lung carcinoma; small cell lung carcinoma; bladder carcinoma; epithelial carcinoma; glioma; astrocytoma; medulloblastoma; craniopharyngioma; ependymoma; pinealoma; hemangioblastoma; acoustic neuroma; oligodendroglioma; meningioma; melanoma; retinoblastoma; and leukemias. In a preferred embodiment, the cancer is selected from the group comprising: breast; cervical; prostate; ovarian, colorectal, lung, lymphoma, and leukemias, and/or their metastases.

As used herein, the term “inflammatory disorder” should be taken to mean an immune-mediated inflammatory condition that affects humans or mammals and is generally characterised by dysregulated expression of one or more cytokines. Examples of inflammatory disorders include skin inflammatory disorders, inflammatory disorders of the joints, inflammatory disorders of the cardiovascular system, certain autoimmune diseases, lung and airway inflammatory disorders, intestinal inflammatory disorders. Examples of skin inflammatory disorders include dermatitis, for example atopic dermatitis and contact dermatitis, acne vulgaris, and psoriasis. Examples of inflammatory disorders of the joints include rheumatoid arthritis. Examples of inflammatory disorders of the cardiovascular system are cardiovascular disease and atherosclerosis. Examples of autoimmune diseases include Type 1 diabetes, Graves disease, Guillain-Barre disease, Lupus, Psoriatic arthritis, and Ulcerative colitis. Examples of lung and airway inflammatory disorders include asthma, cystic fibrosis, COPD, emphysema, and acute respiratory distress syndrome. Examples of intestinal inflammatory disorders include colitis and inflammatory bowel disease. Other inflammatory disorders include cancer, hay fever, periodontitis, allergies, hypersensitivity, ischemia, depression, systemic diseases, post infection inflammation and bronchitis.

As used herein, the term “neurodegenerative disease” should be taken to include motor neurone disease; prion disease; Huntington's disease; Parkinson's disease; Parkinson's plus; Tauopathies; Chromosome 17 dementias; Alzheimer's disease; Multiple sclerosis (MS); hereditary neuropathies; and diseases involving cerebellar degeneration.

As used herein, the term “liver disease” should be taken to mean a disease characterised by liver dysfunction that causes illness. including cirrhosis, hepatitis, liver failure, alcohol or drug-related liver disease, chronic liver disease, jaundice, genetic disorders of the liver (i.e. AAT deficiency), fibrosis and hepatocellular carcinoma.

As used herein, the term “immune disorders” refers to a disease of the immune system or a part of the immune system, and includes certain metabolic diseases (i.e. Type I and Type II diabetes), arthritic (i.e. rheumatoid arthritis), lupus, immune system disorders (i.e. severe combined immune deficiency, temporary acquired immune deficiency, AIDS and HIV), diseases characterised by an over active immune system (asthma, eczema, allergic rhinitis), and autoimmune diseases (i.e. Type 1 diabetes, rheumatoid arthritis, lupus, MS, inflammatory bowel disease, Guillain-Barre syndrome, psoriasis, Graves' disease, and vasculitis).

In the context of treatment and effective amounts as defined above, the term subject (which is to be read to include “individual”, “animal”, “patient” or “mammal” where context permits) defines any subject, particularly a mammalian subject, for whom treatment is indicated. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; and rodents such as mice, rats, hamsters and guinea pigs. In preferred embodiments, the subject is a human.

“Cyclodextrin” refers to a family of compounds made up of sugar molecules bound together in a ring. They are composed of five or more α-D-glucopyranoside units linked 1->4. Examples of cyclodextrins include alpha-cyclodextrin (6-membered sugar ring molecule), beta-cyclodextrin (7-membered sugar ring molecule) and gamma-cyclodextrin (8-membered sugar ring molecule). The term includes modified cyclodextrins and non-modified cyclodextrins. Examples of modified cyclodextrins are well known in the literature and are described for example in EP 1287039, U.S. Pat. No. 7,786,095; EP 2303929, and US 2011/0124103.

“Amphiphile” refers to a compound or molecule possessing both hydrophilic and lipophilic groups. Examples include amphiphilic cyclodextrins, hydrocarbon-based surfactants, phospholipids and glycolipids. By virtue of the ability of amphiphiles of differing structures to associate with each another, forming bilayers, micelles, vesicles and nanoparticles of liquid crystalline structure, an amphiphile can associate with amphiphilic cyclodextrins.

“Complexing amphiphile” refers to an amphiphile which can associate with a cyclodextrin-nucleic acid conjugate of the invention to form a nanoparticulate molecular complex in aqueous solution. Examples are amphiphiles derived from the lipid 1,2-distearoyl-sn-glycero-3-phosphoethanolamine (DSPE) such as 1,2-distearoyl-sn-glycero-3-phosphoethanolamine-N-[amino(polyethylene glycol)-2000] ammonium salt.

“Amphiphilic cyclodextrins” are well known in the literature are typically include cyclodextrins that are functionalised with both a hydrophilic and a lipophilic group. Examples of amphiphilic cyclodextrins are described in US2006148756 and US2011124103. Other examples of amphiphilic cyclodextrins are described in Bilensoy et al (Expert Opin Drug Deliv 2009 November; 6(11)), Alejandro Daz-Moscoso, et al. (Polycationic Amphiphilic Cyclodextrins for Gene Delivery: Synthesis and Effect of Structural Modifications on Plasmid DNA Complex Stability, Cytotoxicity, and Gene ExpressionChem. Eur. J. 2009, 15, 12871-12888), and Pflueger et al. (Cyclodextrin-based facial amphiphiles: assessing the impact of the hydrophilic-lipophilic balance in the self-assembly, DNA complexation and gene delivery capabilities” Org Biol Chem 2016. DOI: 10.1039/c6ob01882c).

“Cyclodextrin-nucleic acid conjugate” refers to a cyclodextrin having at least one nucleic acid molecule (generally an RNA molecule) conjugated, typically via its 3′ terminal base, to a glucosyl unit of the cyclodextrin at a glucosyl 6-, 2- or 3-position. Different conjugates falling within the scope of the invention, and methods of producing the conjugates are described below.

“Nucleic acid” refers to a polymer made up of nucleotide monomers, which typically are DNA or RNA molecules. Examples of DNA molecules include DNA-based nucleic acid constructs, for example genes, gene constructs, transcriptional regulatory elements, operons, promotors, anti-sense oligonucleotides, repressors and the like. Examples of RNA molecules include mRNA (messenger RNA), siRNA (small interfering RNA), shRNA (small hairpin RNA), gRNA (guide RNA), crRNA (RNA which forms an active complex with Cpf1 endonuclease), miRNA (micro RNA), aptamers, CRISPR-Cas9, or analogs thereof. In one embodiment, the nucleic acid molecule is a RNA molecule. The mechanism and applications of RNA interference are described in Microbiol Mol Biol Rev v67(4); 2003. A database of siRNA molecules is provided in Chalk et al, Nucleic Acids Res. 2005 Jan. 1:33. A database of modified siRNA molecules is provided in Nature; Scientific Reports 6, Article No: 20031.

“Targeting ligand” refers to a moiety that can be attached to the cyclodextrin, generally at the R₁, R₂ and R₃ position, that confers targeting functionality to the conjugate. The targeting ligand may target the conjugate to a particular cell type (i.e. by targeting a cell surface protein that is specific to a particular cell type), and/or may confer cell permeability functionality to the conjugate. In one embodiment, the targeting ligand is a cancer cell targeting ligand, for example specific to solid tumor cancel cells or cells of a haematological malignancy. Examples of solid tumors include sarcomas, carcinomas and lymphomas. In one embodiment, the cancer cell is a prostate cancel cell, a cancer ell having a metastatic phenotype, or a prostate cancer stem cell. In one embodiment, the targeting ligand is specific for the brain, central nervous system or peripheral nervous system. Examples of the targeting ligands from the literature include folate (Evans et al UP, 532: 2017), anisamide (Evans et al. Mol Pharm 14: 2017), antibody fragments (Guo et al. Mol Pharm 114: 2017), PEG (Godhino et al. IJP 473: 2014), fusogenic peptide Gala (Evans et al. IJP 2017), the cell penetrating peptide octaarginine (O'Mahony et al. Pharm Res 30: 2013), Rabies Virus Glycoprotein (RVG) (Gooding et al. EJPS 71: 2015), galactosylated groups (lactose, galactose, GalNAc (N-acetyl galactosamine)-derivatised group) (McMahon et al. JPP 2012), and the brain-targeting ligands/techniques described in Malhotra et al (Molecular Biosystems 2015 “RNAi therapeutics for brain cancer: current advancements in RNAi delivery strategies”—especially Table 3 on pages 13-15).

“Lipophilic group” refers to a group containing a predominance of hydrocarbon over other elements so as to influence the solubility or self-association of the molecule in water, for example a chain of the form CH₃(CH₂)ₙ, with n>4.

“Hydrophilic group” refers to one of the following: a charged anionic group such as carboxylate RCO₂⁻, sulfate RSO₄⁻, phosphate; a charged cationic group such as ammonium RNH₃⁺; an uncharged polar group such as OH, oligoethyleneglycol.

“Amphiphilic molecules” have both lipophilic groups and hydrophilic groups. Amphiphiles may have several lipophilic and/or several hydrophilic groups. Proteins and amphiphilic cyclodextrins are examples of such molecules.

“Aliphatic group” refers to a non-aromatic hydrocarbon chain which may be straight-chain, branched-chain, or cyclic. Cyclic aliphatic groups are known as alicyclic groups. Generally, the aliphatic group has 2-18 carbon atoms.

“Aromatic group” refers to a monocyclic or polycyclic structure of alternating sigma bonds and delocalised pi electrons. Examples of aromatic groups include phenyl, benzyl, azole.

“Heterocyclic group” refers to a cyclic group of atoms, at least two of which are different. Examples of heterocyclic groups include oxane and pyran (carbon and one oxygen), piperidine and pyridine (carbon and one nitrogen), 1,3,5-triazine (three carbons and three nitrogens).

“Polyaminoacid” refers to a polymer of one type of aminoacid, as distinct from a peptide or protein which are macromolecules composed of different aminoacids, linked by amide bonds.

“Oligosaccharide” refers to an saccharide oligomer containing a small number (typically three to ten) simple sugars (monosaccharides) which may be the same or different. An oligosaccharide used as a targeting ligand may be antennary (many-branched) to exploit the cluster effect (augmentation of ligand binding). Oligosaccharide binding may be mimicked by employing analogues such as branched polyethyleneglycol (PEG) where each branch terminates in a monosaccharide ligand.

“Steroid” refers to a compound possessing the atomic skeleton of cyclopenta[a]phenanthrene or a skeleton derived therefrom by one or more conceptual bond scissions or ring expansions or contractions. Methyl groups are normally present at carbon-10 and carbon-13. An alkyl side chain may also be present at carbon-17.

“Polyethylene glycol (PEG) group”, also termed polyethylene oxide group, refers to a polyether group of structure R′—(O—CH₂—CH₂)ₙ—OR. The core R group may have a number of PEG groups attached to it, and the R′ group may have a number of functions, for example as a glycosyl group acting as a targeting ligand.

“Glycosyl” refers to any structure obtained by removing the hydroxy group from the hemiacetal function of a monosaccharide or, by extension, of a lower oligosaccharide.

“Glycopeptide” refers to peptides that contain carbohydrate moieties covalently attached to the side chains of the amino acid residues that constitute the peptide.

The term “linker” refers to the bonds or organic moieties (X₁, X₂, X₃) at the 6- or 1- or 2-positions respectively that connect the cyclodextrin to the RNA or to lipophilic, polar or targeting ligands. In one embodiment of the invention, the linker is selected from the group comprising a simple covalent bond or an atom or radical with a valency of at least 2. In this embodiment of the invention, the linker groups comprise ethers, esters, amides, carbonates, ketones, thioethers, thioesters, thioketones, sulfanyl, disulfide, sulfonyl, sulfoxy, sulfones, or a chain of atoms, such as, but not limited to, hydrocarbon groups which are optionally substituted or unsubstituted, contain optional heteroatoms, cyclic or acyclic chains, in which one or more methylene groups can be interrupted or terminated by O, S, S(O), SO2, NR, C(O), substituted or unsubstituted aryl, substituted or unsubstituted heteroaryl, substituted or unsubstituted heterocyclic; where R is hydrogen, acyl, aliphatic or substituted aliphatic. In one embodiment of the invention, the linker is a PEG (polyethylene glycol) group which is a straight chain or branched PEG with one or more of a glycosyl, an oligosaccharide, a peptide, a glycopeptide, a protein, an antibody or other targeting group attached thereto. The advantage of the PEG group is that it increases the systemic circulation time of the nanoparticulate formulation thus ensuring better delivery. A further advantage of the PEG group is that its length can be chosen so that it acts as a spacer group positioning the targeting group at the optimum distance from the macrocycle for binding to a receptor. In one embodiment, the linker attaching the cyclodextrin to the RNA consists in part of a cleavable group which is cleaved under a set of conditions encountered en route to effective therapeutic activity, resulting in removal of the cyclodextrin moiety for the purpose of improving delivery of the RNA over the remainder of the therapeutic delivery pathway. A cleavable linking group is one which, in the environment of the target cell or within the target cell is cleaved to release the two parts that the linker is holding together. In a preferred embodiment, the cleavable linking group is cleaved at least 10 times or more, preferably at least 100 times faster in the target cell or under a first reference condition (which can, e.g., be selected to mimic or represent intracellular conditions) than in the blood of a subject, or under a second reference condition (which can, e.g., be selected to mimic or represent conditions found in the blood or serum). Redox cleavable groups include for example the disulphide group (—S—S—). Acid-cleavable linking groups are cleaved in an environment of about pH 6.5 or lower such as that in the extracellular environment of tumours, or by agents such as enzymes that can act as a general acid. In a cell, organelles of particularly low pH such as endosomes (pH 5.5-6.0) and lysosomes (pH around 5.0) can provide a cleaving environment for such groups. Examples of acid-cleavable groups include but are not limited to hydrazones and esters. Acid-cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl, pentyl or t-butyl. Ester-based cleavable linking groups are cleaved by enzymes such as esterases and amidases in cells. Examples of ester-based cleavable linking groups include but are not limited to esters of alkylene, alkenylene and alkynylene groups. Phosphate-based cleavable groups are cleaved by agents such as phosphatases. Peptide-based cleavable groups are cleaved by enzymes such as peptidases (which can be substrate-specific) and proteases. Peptide bonds can be used when targeting cell types rich in peptidases, such as liver cells.

As used herein, the term “gene therapy” is used to define a therapeutic intervention in which a genetic lesion (e.g. a genetic lesion giving rise, directly or indirectly, to a disease phenotype) is repaired. Gene therapy according to the invention may therefore define therapeutic interventions in which a mutant allele giving rise to a disease phenotype is replaced by an allele which is not associated with the disease phenotype, for example being replaced by a wild-type allele.

In this context, the term wild-type is a term of the art which defines one or more typical forms of a gene as it occurs in nature as distinguished from mutant or variant forms, and in particular mutant or variant forms associated with a disease or disorder.

The term “gene therapy” may also define a therapeutic intervention in which a mutant gene exhibiting a dominant disease phenotype is insertionally inactivated (and thereby “knocked-out” or “silenced”) by replacing a portion of the mutant gene with an exogenous DNA insert which prevents expression of the mutant gene (e.g. via frameshift or nonsense mutations).

The term “proteostatic disease” is a term of art used to define a set of diseases mediated, at least in part, by deficiencies in proteostasis. The term therefore covers aggregative and misfolding proteostatic diseases, including in particular neurodegenerative disorders (e.g. Parkinson's disease, Alzheimer's disease and Huntington's disease), lysosomal storage disorders, diabetes, emphysema, cancer and cystic fibrosis.

As used herein, the term “neoplasia” is used sensu stricto to define diseases involving the abnormal proliferation of neoplastic cells. The term includes benign, pre-cancerous and malignant neoplasia (as defined above) and is used synonymously with the term “proliferative disorder”. The terms “proliferative disorder” and “neoplasia” may be used herein as synonyms to define a class of diseases which involve the pathological growth of cells in vivo.

### BRIEF DESCRIPTION OF THE FIGURES

The invention will be better understood from a description of some examples, given with reference to the accompanying figures:

FIG. 1: Scheme of synthesis of (A) a cyclodextrin-RNA maleimide-linked conjugate (CDmal-RNA) (3), and (B) a cyclodextrin-RNA disulfide-linked conjugate (CDSS-RNA)(5).

FIG. 2: Gel retardation assay of siRNA thiol and CDmal-siRNA conjugate.

FIG. 3: (A) cellular uptake and (B) induced cytotoxicity of CDmal-siRNA (luciferase) conjugate and non-conjugated siRNA, delivered using Lipofectamine 2000 in PC3-Luc cells. The graph shows a representative result (n=4) mean±S.D.

FIG. 4: Gel retardation assays of siRNAs and cyclodextrin conjugates: (a) CDmal-siRNA (luciferase); (b) CDSS-siRNA (luciferase); (c) CDmal-siRNA and CDSS-siRNA (PLK1).

FIG. 5: Reduction in luminescence intensity of PC3-Luc (luciferase) cells on treatment with CD-siRNA (luciferase) conjugates: (A) CDmal-siRNA, (B) CDSS-siRNA, using Lipofectamine 2000 for delivery. The graph shows a representative result of (n=6) mean±S.D. ***P<0.001 was considered highly significant based on Tuckey's posthoc analysis.

FIG. 6: Gene knockdown in PC3 cells with CD-siRNA (PLK1) conjugates (50 nM), delivered using cyclodextrin vector heptakis[2-O—(N-(3″-aminopropyl)-1′H-triazole-4'-yl-methyl)-6-dodecylthio]-β-cyclodextrin trifluoroacetate at mass ratio vector:siRNA 20:1. The graph shows a representative result of (n=4) mean±S.D. **P<0.01 and ***P<0.001 were considered highly significant based on Tuckey's posthoc analysis.

FIG. 7: Gene knockdown in (A) U87 (glioblastoma cells) and (B) DU145 (prostate cancer) cells by CDmal-siRNA (PLK1) conjugate (100 nM), complexed with adamantyl-PEG (Ad-PEG) or -PEG-RVG or -PEG-anisamide, and delivered using chitosan polymer at mass ratios (chitosan:conjugate) 10 and 1. The graph shows a representative result of (n=4) mean±S.D. **P<0.01 and ***P<0.001 were considered highly significant based on Tuckey's posthoc analysis.

FIG. 8: Synthesis of 2-pegylated 6-dodecylthio-cyclodextrin siRNA conjugate (7) from 2-pegylated 6-dodecylthio-cyclodextrin amine (6).

FIG. 9: Gel retardation assay of 2-pegylated 6-dodecylthio-cyclodextrin siRNA (PLK1) conjugate.

FIG. 10: Gene knockdown in PC3 cells by 2-pegylated 6-dodecylthio-cyclodextrin siRNA (PLK1) conjugate (100 nM) at 24 hrs: without vector; delivered with cyclodextrin vector heptakis[2-O—(N-(3″-aminopropyl)-1′H-triazole-4′-yl-methyl)-6-deoxy-6-dodecylthio]-β-cyclodextrin trifluoroacetate at mass ratio MR 20; and delivered with Lipofectamine vector. The graph shows a representative result of (n=3) mean±S.D. **P<0.01 and ***P<0.001 were considered highly significant based on Tuckey's posthoc analysis.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### EXAMPLES

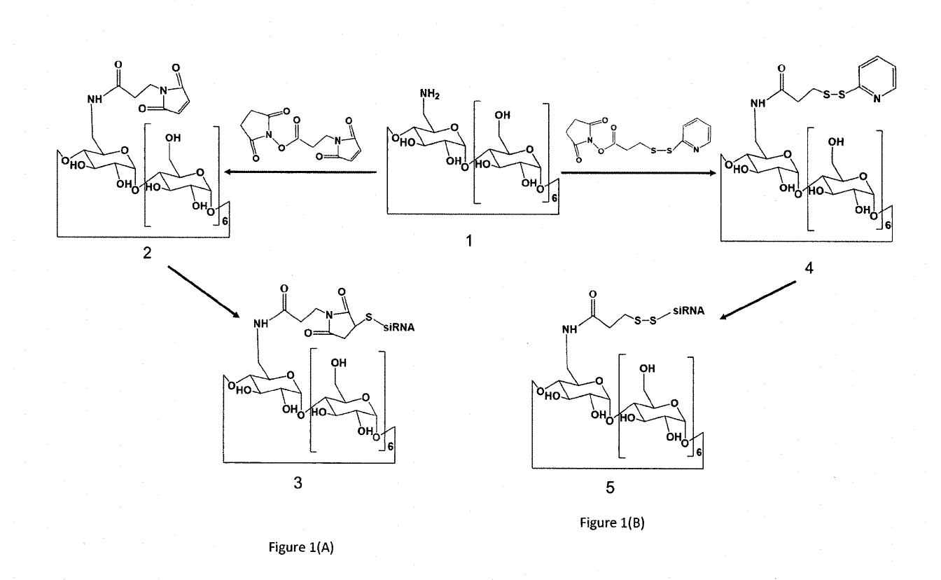

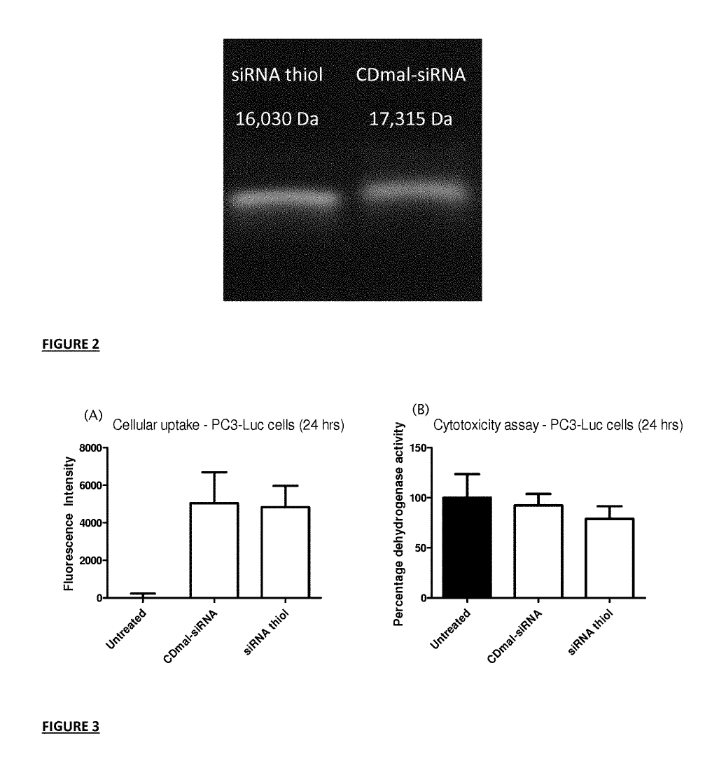

Example 1 illustrates the synthesis of conjugates of β-cyclodextrin with an RNA according to the schemes in FIG. 1.

Example 2 illustrates luciferase gene knockdown in PC3-Luc prostate cancer cells by CD-siRNA conjugates delivered using Lipofectamine™ Example 3 illustrates PLK1 gene knockdown in prostate cancer cells by CD-siRNA conjugates using amphiphilic polycationic cyclodextrin vector heptakis[2-O—(N-(3″-aminopropyl)-1′H-triazole-4 '-yl-methyl)-6-dodecylthio]-β-cyclodextrin trifluoroacetate as delivery agent.

Example 4 illustrates gene knockdown by a receptor-targeted (RVG peptide) formulation comprising: a PLK1 siRNA conjugate (CDmal-siRNA conjugate) complexed with adamantyl-PEG500-RVG by molecular inclusion, charge-neutralised by chitosan polymer at MR 1 and 10.

Example 5 illustrates the synthesis of 2-pegylated 6-dodecylthio-cyclodextrin siRNA conjugate, its gel retardation assay and PLK1 gene knockdown in PC3 cells.

### Example 1

### Synthesis of β-Cyclodextrin-RNA Conjugates According to FIG. 1

### General Experimental Procedures for Chemical Synthesis:

Chemical materials. All chemicals were purchased from the Aldrich chemical company and were used without further purification unless noted.

Triphenylphosphine was recrystallised from ethanol and dried under high vacuum for 6 hours at 50° C. before use.

β-cyclodextrin was dried for 12 hours at 100° C. under high vacuum before use.

DMF was purchased in Sureseal bottles over molecular sieves and stored under nitrogen. Chromatography. Thin-layer chromatography was performed on aluminium-backed plates of Merck Silica Kieselgel 60 F₂₅₄. Carbohydrate R_{f} values were found by dipping the plates in a 5% sulfuric acid in ethanol solution and heating with a heat gun; or in caesium sulfate stain (21 g (NH₄)₆Mo₇O₂₄, 1 g Ce(SO₄)₂, 1 L H₂O, 31 mL conc. H₂SO₄) followed by charring; or developed in an iodine tank containing iodine mixed with sand.

Flash chromatography was carried out on Merck Kieselgel 60 0.04-0.063 mm.

NMR Spectroscopy. ¹H NMR spectra were recorded using Varian 300, 400 and 500 MHz spectrometers at 25° C. unless otherwise stated.

¹³C NMR spectra were recorded on Varian 75, 100 and 125 MHz spectrometers at 25° C. unless stated otherwise.

The atom-numbering scheme is indicated in claim 1, and atoms listed in the NMR data are identified by the atomic groups in which they occur.

Mass Spectrometry. MALDI-TOF analyses were performed on a Perseptive (Framingham, Mass.) Voyager STR instrument equipped with delayed extraction technology. Ions were formed by a pulsed UV laser beam (nitrogen laser, 337 nm) and accelerated through 24 kV. Samples were diluted in CHCl₃ and mixed 1:1 v/v with the matrix solution obtained by dissolving 2,5-dihydroxybenzoic acid (DHB) in CH₃OH-0.1% trifluoroacetic acid-CH₃CN (1:1:1 by volume) at a concentration of 30 mg/mL Exactly 1 mL of this mixture was deposited onto a stainless steel 100-sample MALDI plate and allowed to dry at room temperature before running the spectra in the positive polarity.

### 6-Deoxy-6-(3'-(N-maleimido)-propionamido)-β-cyclodextrin (2)

6-Amino-6-deoxy-β-cyclodextrin (1) was synthesized according to a literature method (Weihua, T., Siu-Choon, N., Nature Protocol, 2008, 3: 691-695). Compound 1 (115 mg, 0.10 mmol) was dissolved in deionised water (6 mL) and 3-(N-maleimido)-propionic acid N-hydroxysuccinimide ester (54 mg, 0.20 mg) was added in DMF (1.4 mL). The mixture was stirred for 3 h at room temperature, then the product (2) was precipitated with acetone and washed several times with acetone (yield 101 mg, 79%).

H1-NMR (400 MHz, DMSO-d₆): δ 6.97 (s, 2H), 5.73 (m, 14H), 4.82 (s, 7H), 4.67 (m, 7H), 3.82-3.47 (m, 30H).

13C-NMR (101 MHz, DMSO-d₆): δ 171.1, 134.9, 102.4, 82.0, 73.5, 72.9, 72.5, 60.4.

ESI-MS (m/z): 1307.66 [M+Na]⁺.

Molecular weight: 1285.11894; molecular formula: C₄₉H₇₆N₂O₃₇

### 6-deoxy-6-(3′-(2″-pyridyldithio)-propionamido)-β-cyclodextrin (4)

6-Amino-6-deoxy-β-cyclodextrin (FIG. 1, 1) (45 mg, 0.04 mmol) was dissolved in deionised water (4 mL) and 3-(2′-pyridyldithio) propionic acid N-hydroxysuccinimide ester (25 mg, 0.08 mg) was added in DMF (1.2 mL). The mixture was stirred for 3 h at room temperature and the product (4) was precipitated with acetone and washed several times with acetone (46 mg, 86%).

1H NMR (400 MHz, DMSO-d6) δ 8.43 (d, J=4.7 Hz, 1H), 7.91-7.64 (m, 2H), 7.33-7.11 (m, 1H), 6.00-5.48 (m, 6H), 4.96-4.67 (m, 3H), 4.43 (s, 3H), 3.87-3.43 (m, 10H), 3.44-3.08 (m, 25H, overlap with D₂O).

13C NMR (101 MHz, DMSO-d6) δ 170.49, 159.64, 149.98, 138.25, 119.53, 102.37, 73.43, 72.85, 72.46, 60.34, 34.50.

ESI-MS (m/z): 1328.66 [M−H]⁻

Molecular weight: 1331.27672; molecular formula: C₅₀H₇₈N₂O₃₅S₂

### Full Reduction of Thiol-Modified siRNA

Two thiol-modified siRNAs with sequences against luciferase gene and PLK1 gene were obtained from Gene Link (NY, USA). The siRNA targeted against luciferase gene had the following sequence: sense strand, 5′-GAA GUG CUC GUC CUC GUC CCC C dT dT-(Thiol-M C6-D) 3′ MW 7,979 Da [SEQ ID 1]; antisense strand, 5′-GGG GGA CGA GGA CGA GCA CUU C dT dT-3′ MW 7,514 Da [SEQ ID 2]. The siRNA targeted against PLK1 had the following sequence: sense strand, 5′-AGA mUCA CCC mUCC UmUA AAmU AUU dT dT-(Thiol-M C6-D) 3′ [SEQ ID 3], antisense strand, 5′-UAU UUA AmGG AGG GUG AmUC UUU-3′ [SEQ ID 4], with a total molecular weight of 13,558 Da. The PLK1 siRNA had 2′-O-methyl “m” modifications on its nucleotides to protect it from serum nucleases.

Thiol-modified luciferase siRNA, or thiol-modified PLK1 siRNA, was fully reduced as follows: 1 mL of 3% TCEP (tris(2-carboxyethyl)phosphine) solution was freshly prepared by adding 30 μL of TCEP to 970 μL of sterile RNAse-free water. From this solution, 4004 was added to the lyophilized thiolated siRNA. The solution was left at room temperature for 1 hr, then 50 μL of 3M sodium acetate solution at pH 5.2 was added before again vortexing. Absolute ethanol (1.5 mL) was added to the solution which was vortexed briefly and stored at −80° C. for 20 minutes. The sample was centrifuged at 12 K rpm for 10 minutes, the supernatant was decanted and the pellet air dried. The pellet was dissolved in 200 μL of sterile phosphate-buffered saline (PBS), pH 7.2. Total concentration of the siRNA in 200 μL was determined by measuring absorbance at 260 nm, using a Nanodrop spectrophotometer.

### Conjugation of siRNA with β-Cyclodextrin Through a Maleimide Link

A 10-fold molar excess of 6-deoxy-6-(3′-(N-maleimido)-propionamido)-β-cyclodextrin (FIG. 1, 2) in 50 μl of RNAse-free water was added to a solution of a thiol-modified siRNA (100 μL in PBS, pH 7.2). The reaction solution was vortexed gently and incubated at room temperature overnight to obtain the CDmal-siRNA conjugate (FIG. 1, 3). Excess of the cyclodextrin was removed by using Amicon centrifugal filters (MW 3000) (Millipore, Ireland), centrifuged at 5000 rpm and the solution was reconstituted with 100 μL of RNAse-free water. The total CDmal-siRNA conjugate concentration was determined by measuring absorbance at 260 nm using a Nanodrop spectrophotometer.

### Conjugation of siRNA with β-Cyclodextrin Through a Disulphide Link

A 10-fold molar excess of 6-deoxy-6-(3′-(2″-pyridylthio)-propionamido)-β-cyclodextrin (FIG. 1, 4) in 100 μl of DMSO was added to a solution of a thiol-modified siRNA (100 μL in PBS buffer pH 7.2). The pH was adjusted to 4-5 with 1M HCl and 100 mM sodium acetate. The reaction mixture was gently vortexed and incubated at 35° C. overnight to obtain the CDSS-siRNA conjugate (5). Excess of the cyclodextrin was removed by using Amicon centrifugal columns (MW 3000) (Millipore, Ireland) centrifuged at 5000 rpm, and the solution was reconstituted with 100 μL of RNAse-free water. Total CDSS-siRNA concentration was determined by measuring absorbance at 260 nm using a Nanodrop spectrophotometer.

### Gel Retardation Assay of CD-siRNA Conjugates

Agarose (1% w/v) (Sigma, MO, USA) solution was prepared with 1× Tris borate EDTA (TBE) buffer to which 6 μL of SafeView™ (NBS Biologicals Ltd, England) was added to visualize siRNA bands under UV light.

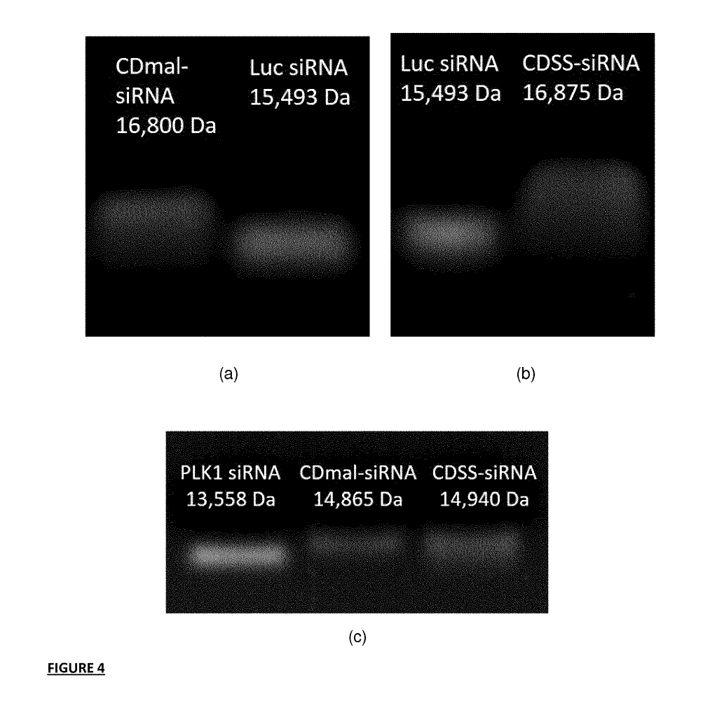

The unconjugated and conjugated siRNA (36 μL) were each mixed with 10× Blue Juice gel loading buffer (4 μL) (Invitrogen, Ireland). Electrophoresis was carried out at 90 V for 45 minutes in TBE buffer. The siRNA bands were visualized by UV using DNR Bioimaging Systems MiniBis Pro and Gel capture US B2 software. FIGS. 4a and 4b show luciferase siRNA conjugated to cyclodextrin maleimide (CDmal) and disulfide (CDSS) respectively. FIG. 4c shows PLK1 siRNA conjugated to CDmal and CDSS.

### Electron-Spray Ionisation (ESI) Mass Spectroscopy of CD-siRNA Conjugates

The samples for ESI were prepared at a concentration of 40-50 μM in 0.1M TEAA (triethanolamine acetate) buffer, pH 7. A C18 column was used with the following mobile phase: solvent A, 15 mM TEA with 400 mM HFIP (hexafluoro-2-propanol) (pH 7.9), measured by weight; solvent B, 50% solvent A with 50% methanol (v/v). ESI analysis shows two peaks, for sense and antisense strands of the siRNA. The molecular weights of sense and antisense strands of luciferase siRNA as stated by the supplier (Gene Link, USA) were 7979 and 7514. ESI results (Table) showed conjugation of luciferase siRNA (sense strand) with CD-mal and CD-SS, resulting in increased molecular weights to 9261.63 and 9325.54.

| | Mass of sense | Theoretical mass |
|---|---|---|
| Sample | strand by ESI | of sense strand |
| Luciferase siRNA | 7725.54 | 7979 |
| CDmal-siRNA conjugate | 9261.63 | 9261.12 |
| CDSS-siRNA conjugate | 9325.54 | 9310.28 |

### Example 2

### Luciferase Gene Knockdown in Prostate Cancer Cells by CD-siRNA Conjugates Delivered Using Lipofectamine™

The human prostate cancer cells (PC3-Luc) (Sigma, Germany), over-expressing luciferase gene were grown in RPMI 1640 cell culture medium, supplemented with 10% foetal bovine serum (FBS) (Sigma, Germany) and 1% of 2 mM L-glutamine (GIBCO, UK). For passaging, 0.05% trypsin-EDTA (GIBCO, United Kingdom) was used. The cells were maintained in a humidified chamber at 37° C. with 5% CO₂.

Cellular delivery of luciferase CD-siRNA conjugates was performed using Lipofectamine 2000 (Lf2000) (Sigma, USA): 50 nM of unconjugated or conjugated siRNA was delivered using Lf2000 with final vector:siRNA ratio 1 μL Lf2000:20 pmol siRNA, as recommended by the supplier. 10,000 PC3-Luc cells/well were seeded in a 96-well white-opaque plate and transfection was carried out after 24 hours of seeding. The cells were incubated with the treatment samples for 4 hours, after which the medium was replaced with complete growth medium. The percentage of luciferase gene knockdown (protein suppression) was analyzed at 48 hours by measuring the luminescence intensity using a multiplate reader (Perkin Elmer—Wallac Victor 2T_{M} 1420 multilabel counter).

FIG. 5 shows the reduction in luminescence intensity of PC3-Luc (luciferase) cells on treatment with luciferase siRNA and its cyclodextrin conjugates, using Lipofectamine as delivery agent: (A) CDmal-siRNA; (B) CDSS-siRNA. Conjugation to cyclodextrin does not reduce the silencing efficiency of the siRNA. The luciferase protein suppression achieved for luciferase siRNA was highly significant (p<0.0001) in comparison with the scrambled siRNA.

### Example 3

### PLK1 Gene Knockdown in Prostate Cancer Cells by CD-siRNA Conjugates Using Amphiphilic Polycationic Cyclodextrin Vector heptakis[2-O—(N-(3″-aminopropyl)-1′H-triazole-4′-yl-methyl)-6-dodecylthio]-β-cyclodextrin trifluoroacetate as Delivery Agent

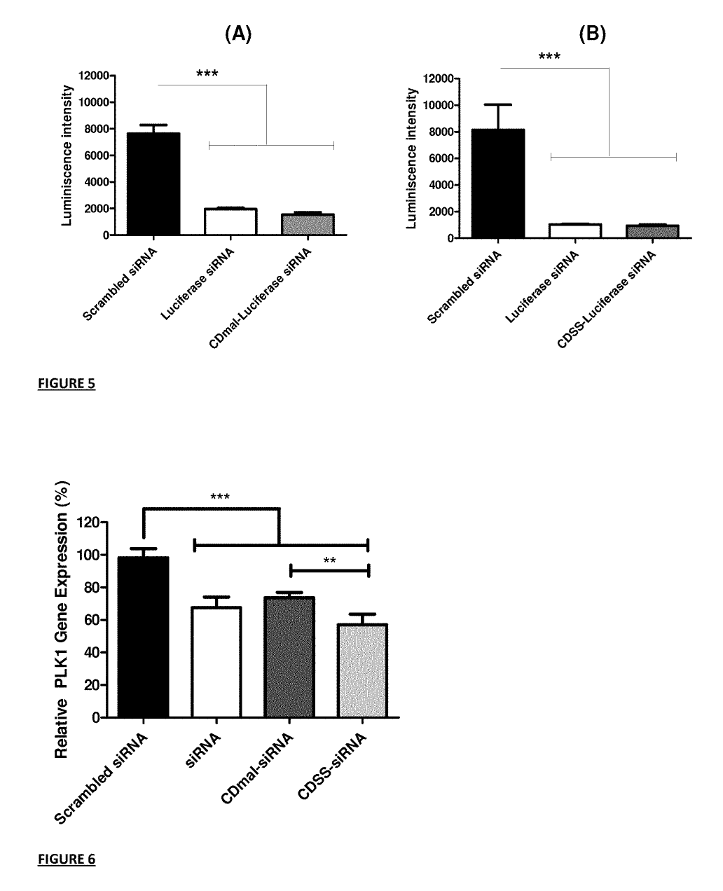

PC-3 cells (100,000 cells/well) were seeded in a 24-well plate. CD-siRNA (PLK1) conjugates were delivered, using the cyclodextrin vector, after 24 hours seeding. Concentration of conjugate was 50 nM and cyclodextrin vector was used at a mass ratio to conjugate (MR) of 20. The percentage of PLK1 gene knockdown was analyzed by qRT-PCR to quantify the relative reduction of PLK1 mRNA expression (FIG. 6). Knockdown was comparable to that of unconjugated PLK1 siRNA (P>0.05). The siRNA conjugated through a cleavable (disulfide) linker showed enhanced knockdown compared with that conjugated through a non-cleavable (maleimide) linker (P<0.001).

### Example 4

### Gene Knockdown by a Receptor-Targeted (RVG Peptide) Formulation Comprising: CDmal-siRNA (PLK1) Conjugate Complexed with Adamantyl-PEG500-RVG by Molecular Inclusion, Charge-Neutralised by Chitosan Polymer at MR 1 and 10

Complexation of chitosan with an unmodified scrambled siRNA (FAM labelled) was first optimised to obtain a surface charge for the chitosan.siRNA nanoparticles of 0±5 mV. This ensured that cellular uptake was not enabled by positive surface charge, but by surface-complexed adamantyl-PEG500-RVG targeting U87 cells, and by adamantyl-PEG5000-dianisamide (AA₂) targeting sigma 1 receptors on DU145 cells. Stable nanoparticles with near neutral surface charge were formed at chitosan MR 1 and 10.

Human glioblastoma (brain cancer) cells (U87), and prostate cancer (DU145) cells were grown in DMEM cell culture medium and RPMI 160 cell culture medium respectively, supplemented with 10% foetal bovine serum (FBS) (Sigma, Germany) and 1% 2 mM L-glutamine (GIBCO, United Kingdom). For passaging 0.05% Trypsin-EDTA (GIBCO, United Kingdom) was used. The cells were kept and maintained in a humidified chamber at 37° C. with 5% CO₂.

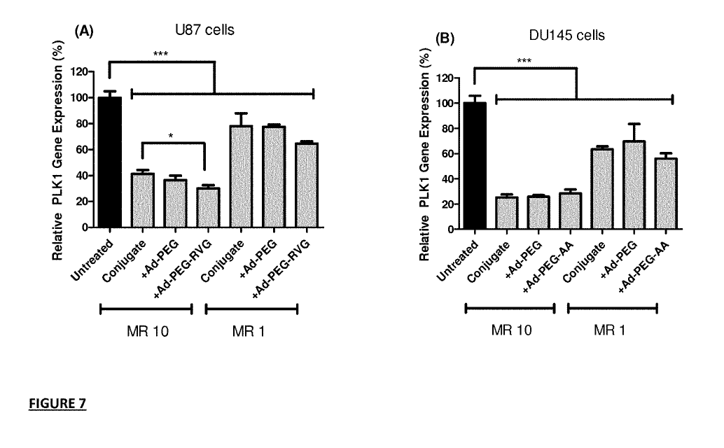

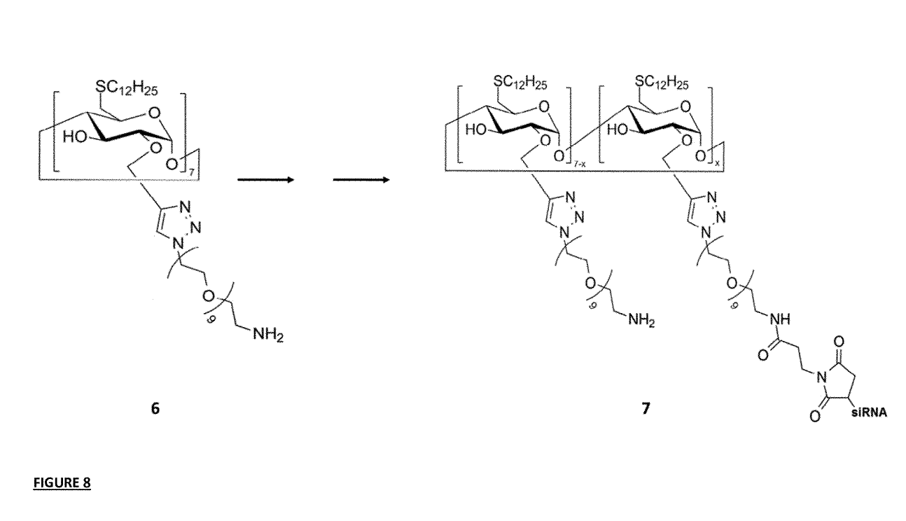

Cellular delivery of CDmal-siRNA (PLK1) conjugate was performed using chitosan polymer at mass ratios chitosan:conjugate 10 and 1. U87 (25,000 cells/well) and DU145 (50,000 cells/well) were seeded in 24-well plates and delivery was carried out after 24 hrs seeding. For delivery, inclusion complexes with adamantyl-PEG and adamantyl-PEG-RVG were formed, prior to complexing the conjugate with chitosan polymer (MR 10 and 1). Concentration of conjugate was 100 nM. The nanoformulations were incubated for 24 hrs and the gene knockdown was analyzed at 48 hrs. The percentage of PLK1 gene knockdown was analyzed by qRT-PCR to quantify the relative reduction of PLK1 mRNA expression in the U87 and DU145 cells (FIG. 7). PLK1 gene knockdown of 30 to 40% (P<0.0001) was obtained at MR1 and 60 to 70% (P<0.0001) at MR10 in U87 cells, with the RVG-targeted formulation being especially significant (P<0.05). Similar knockdown was obtained in DU145 cells: 75% at MR 10 (P<0.0001) and 40 to 50% at MR1 (P<0.0001), although no difference in the receptor targeted formulation was observed.

### Example 5

### Synthesis of 2-pegylated 6-dodecylthio-cyclodextrin siRNA Conjugate, Gel Retardation Assay and PLK1 Gene Knockdown in PC3 Cells

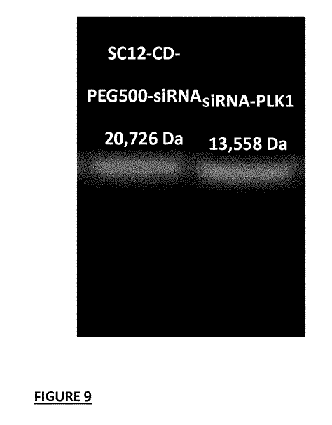

The 2-pegylated 6-dodecylthio-cyclodextrin amine (compound 6, FIG. 8) was synthesized according to the literature method (Gooding M, et al. European Journal of Pharmaceutical Sciences 71 (2015) 80-92.) This cyclodextrin was conjugated to the thiol-modified siRNA using 3-maleimido-propionic acid N-hydroxysuccinimide ester (BMPS) linker as follows (FIG. 8).

Compound 6 (19 mg, 0.003 mmol) and BMPS linker (8 mg, 0.03 mmol) were dissolved in anhydrous DMF (2 mL) and DIEA was added (25 μl, 0.15 mmol). The solution was stirred at room temperature for 48 h under nitrogen. The solution was then evaporated, and the residue dissolved in 1 mL of methanol and purified by gel filtration over Sephadex LH20 (Gooding M, et al, 2015). The resulting maleimide (2 mg), in 100 μl of DMSO, was reacted with 50 μg of thiol-modified siRNA (PLK1) in 200 μl of conjugation buffer (PBS, pH 7.2) at 30° C. on a thermomixer at 900 rpm for 24 hrs. The resulting conjugate (compound 7, FIG. 8) was purified using an Amicon centrifugal filtration column (M.W. 10,000 Da) at 4000 rpm. The sample concentration was measured at 260 nm using a Nanodrop spectrophotometer.

Gel retardation assay was performed to confirm conjugation of the siRNA. Agarose (1% w/v) (Sigma, MO, USA) solution was prepared with 1× Tris borate EDTA (TBE) buffer to which 6 μL of SafeView™ (NBS Biologicals Ltd, England) was added to visualize siRNA bands under UV light. The conjugated and unconjugated siRNA (36 μL) were each mixed with 10× Blue Juice gel loading buffer (4 μL) (Invitrogen, Ireland). Electrophoresis was carried out at 90 V for 45 min in TBE buffer. The siRNA bands were visualized by UV using DNR Bioimaging Systems MiniBis Pro and Gel capture US B2 software. FIG. 9 shows PLK1 siRNA conjugated to 2-pegylated 6-dodecylthio-cyclodextrin using BMPS linker.

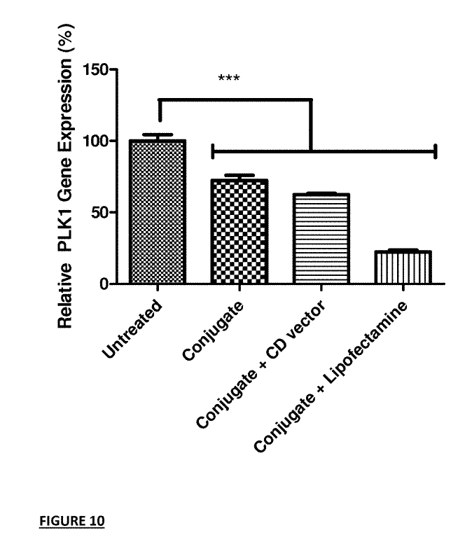

PC3 cells (100,000 cells/well) were seeded in a 24-well plate for 24 h. siRNA (PLK1) conjugates were delivered using the cyclodextrin vector heptakis[2-O—(N-(3″-aminopropyl)-1′H-triazole-4′-yl-methyl)-6-dodecylthio]-β-cyclodextrin trifluoroacetate or Lipofectamine vector. Concentration of conjugate was 100 nM and cyclodextrin vector was used at a mass ratio to conjugate (MR) of 20. PLK1 gene knockdown was measured by qRT-PCR, by quantifying reduction of PLK1 mRNA expression (FIG. 10). Approximately 30% knockdown was obtained with 2-pegylated 6-dodecylthio-cyclodextrin siRNA conjugate alone, which showed that it is capable of self-delivery. Approximately 40% and 80% knockdown was achieved with the cyclodextrin vector and Lipofectamine respectively. The results show that conjugation of an RNA to an amphiphilic cyclodextrin confers improved self-assembly and delivery properties on the RNA.

The invention is not limited to the embodiment hereinbefore described, but may be varied in both construction and detail within the scope of the appended claims.

1. A cyclodextrin-RNA conjugate in which the cyclodextrin molecule is conjugated at its glucosyl 6-, 2- or 3-positions, optionally via a linker, to at least one RNA molecule at the RNA 3′ terminal base.

2. The cyclodextrin-RNA conjugate according to claim 1 in which at least one of the RNA molecules is a siRNA.

3. The cyclodextrin-RNA conjugate according to claim 1 in which the cyclodextrin is an amphiphilic cyclodextrin.

4. An amphiphilic cyclodextrin-RNA conjugate which has the following formula: 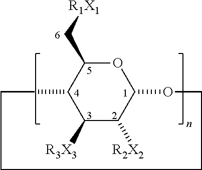
in which:
n equals 6 or 7 or 8, and indicates the number of modified or unmodified glucose units in e cyclodextrin macrocycle which are the same or different, depending on the X- and R-groups;
X₁, X₂, X₃ independently provide linkers and are a simple covalent bond or an atom or radical having a valency of at least two;
R₁, R₂ and R₃ independently are selected from the groups comprising (a) lipophilic groups, (b) polar groups and/or groups capable of hydrogen bonding, (c) targeting ligands, and (d) an RNA molecule, wherein at least one of R₁, R₂ and R₃ is an RNA molecule.

5. The cyclodextrin-RNA conjugate according to claim 4 in which R₁ is a lipophilic group and in which R₂ and R₃ are independently selected from polar groups and/or groups capable of hydrogen bonding.

6. The cyclodextrin-RNA conjugate according to claim 4 in which the cyclodextrin has a primary side that is hydrophilic and comprises the R₁X₁ groups and a secondary side that is lipophilic and comprises the R₂X₂ and R₃X₃ groups, wherein R₁ and one of R₂, R₃ are independently a polar group and/or group capable of hydrogen bonding, and in which the other of R₂, R₃ is a lipophilic group.

7. The cyclodextrin-RNA conjugate according to claim 4 in which the groups that are polar and/or capable of hydrogen-bonding are selected from: H; OH; (CH₂)₂₋₄OH; CH₂CH(OH)CH₂OH; CH₂CH(OH)CH₂NH₂; an amine group; a cationic group; an anionic group; a polyamino acid; a peptide; an oligosaccharide; a polyethylene glycol (PEG) group; and a hydrophilic group.

8. The cyclodextrin-RNA conjugate according to claim 4 in which the targeting ligand is selected from a group comprising: a polyaminoacid, a peptide, an oligosaccharide, a steroid, —P(Y)(Z)O-nucleoside, —P(Y)(Z)O-oligonucleotide, —P(Y)(Z)O-Linker-OP(Y′)(Z′) O-oligonucleotide, a nucleotide, an oligonucleotide, wherein Y, Z, Y′ and Z′ are independently O, or S.

9. The cyclodextrin-RNA conjugate according to claim 4 in which the lipophilic group is selected from an aliphatic chain, an alicyclic, aromatic or heterocyclic group, or combinations thereof.

10. The cyclodextrin-RNA conjugate according to claim 4 wherein X₁, X₂, X₃ are independently selected from an ether, ester, methylene, methylenoxy, ethylene, ethylenoxy, carbonyl, thioether, thioester, thiocarbonyl, sulfanyl, disulfide, sulfonyl, sulfoxy, amido, amino, phosphate, thiophosphate, triazolyl.

11. The cyclodextrin-RNA conjugate according to claim 4 wherein the amine group is selected from the group comprising NH₂, NHR where R is one or more of a methyl group, ethyl group, a branched or dendrimeric group comprising one to ten amine groups or a peptide containing basic amino acids, where one or more of the amino groups is optionally in its protonated form, preferably as a hydrohalide salt or trifluoroacetic acid salt.

12. The cyclodextrin-RNA conjugate according to claim 7, wherein the polyethylene glycol (PEG) group is a straight-chain or branched group with one or more independently of a glycosyl, an oligosaccharide, a peptide, a glycopeptide, a protein, an antibody and/or a targeting ligand attached thereto.

13. The cyclodextrin-RNA conjugate according to claim 4 in which the targeting ligand is selected from folate, anisamide, an antibody fragment, a fusogenic peptide, a cell penetrating peptide, rabies virus glycoprotein (RVG), a galactosylated group such as GalNAc (N-acetyl galactosamine)-derivatised group, and prostate surface membrane antigen.

14. A nanoparticulate molecular complex comprising a cyclodextrin-RNA conjugate according to claim 4.

15. The nanoparticulate molecular complex according to claim 14 which is self-assembling and in which the cyclodextrin-RNA conjugate is an amphiphilic cyclodextrin-RNA conjugate of claim 4.

16. The nanoparticulate molecular complex according to claim 14 additionally comprising a complexity amphiphile.

17. The molecular complex according to claim 14 additionally comprising a complexing amphiphile and in which the complexing amphiphile is an amphiphilic cyclodextrin.

18. The nanoparticulate molecular complex according to claim 14 in which the cyclodextrin-RNA conjugate is complexed with a polycation chosen from the following: a polycationic cyclodextrin, a polycationic lipid, a polycationic oligosaccharide, a polycationic polysaccharide, a polycationic peptide, a polycationic polymer.

19. The nanoparticulate molecular complex according to claim 14, incorporating additionally, by host-guest inclusion or by amphiphilic or electrostatic interaction, a lipophile such as a cholesterol or adamantane derivative and/or other lipid derivative.

20. The nanoparticulate molecular complex according to claim 14, coated, by host-guest inclusion or by amphiphilic or electrostatic interaction, with a polyamine, peptide, protein, oligosaccharide, modified cyclodextrin, polysaccharide, antibody and/or antibody fragment.

21.-36. (canceled)


SEQUENCE LISTING (4 sequences)

SEQ ID NO: 1 - DNA, 24 bp, Artificial Sequence
  source: /note=“Description of Artificial Sequence Synthetic oligonucleotide”
  source: /note=“Description of Combined DNA/RNA Molecule Synthetic oligonucleotide”
gaagugcucguccucguccccctt

SEQ ID NO: 2 - DNA, 24 bp, Artificial Sequence
  source: /note=“Description of Artificial Sequence Synthetic oligonucleotide”
  source: /note=“Description of Combined DNA/RNA Molecule Synthetic oligonucleotide”
gggggacgaggacgagcacuuctt

SEQ ID NO: 3 - DNA, 23 bp, Artificial Sequence
  source: /note=“Description of Artificial Sequence Synthetic oligonucleotide”
  source: /note=“Description of Combined DNA/RNA Molecule Synthetic oligonucleotide”
agaucacccuccuuaaauauutt

SEQ ID NO: 4 - RNA, 21 nt, Artificial Sequence
  source: /note=“Description of Artificial Sequence Synthetic oligonucleotide”
uauuuaaggagggugaucuuu